(19) United States
(12) Patent Application Publication
(10) Pub. No.: US 2010/0041716 A1
(43) Pub. Date: Feb. 18, 2010

(54) NITROXIDES FOR USE IN TREATING OR PREVENTING HYPERCHOLESTEROLEMIA

(75) Inventors: Louis Habash, Newport Coast, CA (US); Clarence Jones, Huntington Beach, CA (US)

Correspondence Address: KNOBBE MARTENS OLSON & BEAR LLP, 2040 MAIN STREET, FOURTEENTH FLOOR, IRVINE, CA, 92614, US

(73) Assignee: MITOS PHARMACEUTICALS, INC.

(21) Appl. No.: 12/295,714

(22) PCT Filed: Nov. 28, 2007
(86) PCT No.: PCT/US07/85814

§ 371 Date: Sep. 29, 2009

Related U.S. Application Data
(60) Provisional application No. 60/873,331, filed on Nov. 29, 2006.

(51) Int. Cl. A61K 31/44; A61K 31/40; A61K 31/421; A61P 7/00

(52) U.S. Cl. 514/357; 514/424; 514/374

(57) ABSTRACT

Pharmaceutical compositions are provided that are useful in treating hypercholesterolemia. The compositions comprise a pharmaceutically acceptable carrier, and an effective therapeutic or prophylactic amount of a nitroxide antioxidant that alters the expression of one or more genes related to hypercholesterolemia. Methods are also provided for the use of the pharmaceutical compositions in the treatment or prevention of hypercholesterolemia. In a preferred embodiment, the nitroxide antioxidant is Tempol (4-hydroxy-2,2,6,6-tetramethylpiperidine-1-oxyl).

### BACKGROUND OF THE INVENTION

1. Field of the Invention

The present invention relates to pharmaceutical compositions useful for treating or preventing hypercholesterolemia, and to methods for using these compositions in treating or preventing hypercholesterolemia.

2. Description of the Related Art

Cholesterol is a lipid particle that circulates in the blood; it is the most common bodily steroid and is important in the creation and maintenance of cell membranes and in the formation of bile, vitamin D, and other steroids. Cholesterol is produced by the liver and is also present in the diet: the average adult synthesizes approximately 1 gram and consumes 0.3 grams per day. Hypercholesterolemia refers to an elevated level of cholesterol in the blood. While this is not itself a disease state, it is associated with other diseases, such as diabetes mellitus and metabolic syndrome; nephritic syndrome; hypothyroidism; anorexia nervosa; and Zieve's syndrome.

Left unchecked, furthermore, hypercholesterolemia can contribute to cardiovascular disease. The excess cholesterol in the blood can contribute to the formation of arterial plaques, which are composed of other fatty and fibrous tissues in addition to cholesterol. The accumulation of such plaques results in atherosclerosis, which can express itself in a number of diseases, such as angina pectoris, myocardial infarction, transient ischemic attacks, cerebrovascular accident/stroke, or peripheral artery disease.

Collectively, diseases associated with hypercholesterolemia and related atherosclerosis are major causes of death in industrialized countries. For example, approximately 1.1 million acute myocardial infarctions (AMI), often caused by atherosclerosis of the coronary arteries, occur every year in the United States. Cerebrovascular accidents cause approximately 200,000 deaths in the United States each year, many of these caused by an atherosclerotic vessel that produces a local thrombosis and a distal embolism of the clot.

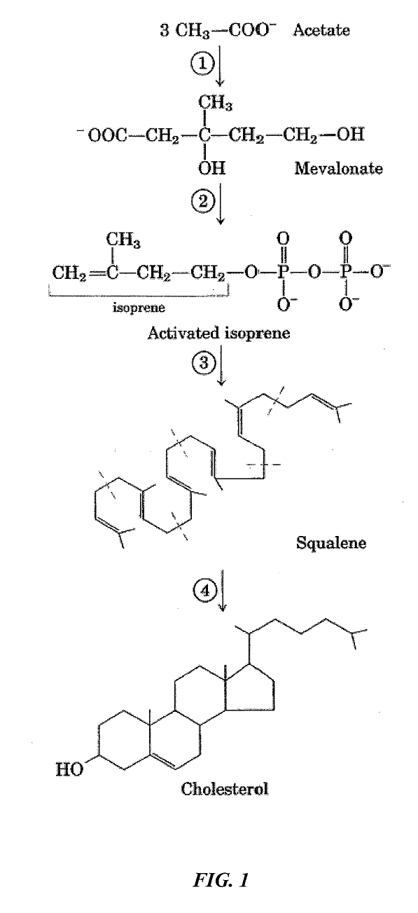

Because hypercholesterolemia is an early-appearing factor that can lead to serious disease, as described above, efforts have been made to combat the condition using pharmaceutical agents. One way in which this has been done is to inhibit one or more of the more than twenty enzymes involved in the cholesterol synthesis pathway so as to limit de novo cholesterol synthesis. An example of this class of drugs is statins, which inhibit the activity of the enzyme 3-hydroxy-3-methylglutaryl CoA reductase. A summary of the stages of cholesterol biosynthesis, omitting many of the individual reaction steps, is shown in FIG. 1. The 3-hydroxy-3-methylglutaryl CoA reductase enzyme participates in the portion of the pathway that leads to the synthesis of the cholesterol precursor mevalonate (thus, it operates in the portion marked (1) in FIG. 1). Although the statins are effective in reducing the amount of mevalonate formed and thus in controlling cholesterol levels, they have exhibited undesirable side effects such as liver dysfunction, general muscle myopathy, skeletal muscle myopathy (rhabdomyolysis), and persistent elevations in serum transaminases. This is attributable to the fact that mevalonate is a precursor not only for cholesterol, but also for other important compounds such as isoprenoids and coenzyme Q10. Thus, the use of statins affects not only downstream levels of cholesterol, but also of isoprenoids and coenzyme Q10, leading to the liver and muscle problems described above.

It would accordingly be desirable to find agents that aid in controlling the amount of cholesterol synthesized in the body without the presence of these side effects. In particular, it would be desirable to find agents that alter the activity level of one or more of the enzymes in the de novo cholesterol synthesis pathway (which accounts for more than 75% of the cholesterol present in the body) that operate at some point downstream from mevalonate synthesis.

Gene therapy offers a potential hypercholesterolemia treatment/prevention alternative to agents such as statins. To this end, it would be desirable to identify genes related to hypercholesterolemia, particularly those encoding enzymes involved in de novo synthesis of cholesterol post-mevalonate synthesis, and to develop methods of altering the expression patterns of those genes so as to prevent the development of hypercholesterolemia or reduce its effects once it has occurred.

### SUMMARY OF THE INVENTION

Pharmaceutical compositions are provided that are useful in preventing and treating hypercholesterolemia. The compositions comprise a pharmaceutically acceptable carrier, and an effective therapeutic or prophylactic amount of an agent that changes the expression pattern of a gene related to hypercholesterolemia. Methods are also provided for the use of the pharmaceutical compositions in the alteration of intracellular levels of hypercholesterolemia-related proteins. In a preferred embodiment, the agent is a nitroxide antioxidant, such as Tempol (4-hydroxy-2,2,6,6-tetramethylpiperidine-1-oxyl).

### DETAILED DESCRIPTION OF THE PREFERRED EMBODIMENT

As described above, a composition and method are disclosed which are useful in treating or preventing hypercholesterolemia. In a preferred embodiment, the agent used to downregulate genes related to hypercholesterolemia is a nitroxide antioxidant. Tempol is a stable nitroxide radical characterized by the chemical formula 4-hydroxy-2,2,6,6-tetramethylpiperidine-1-oxyl that has antioxidative properties. The present applicants have discovered that Tempol also possesses the novel property of altering the expression of genes encoding for proteins associated with the development or progression of hypercholesterolemia (see Table 1 below). Previous therapies have generally not focused on altering the expression patterns of such hypercholesterolemia-related genes.

Tempol accordingly affects the upstream source of implicated proteins by altering the expression of hypercholesterolemia-associated genes.

The use of other nitroxide compounds is also contemplated. According to certain embodiments the nitroxide compound can be selected from the following formulas:

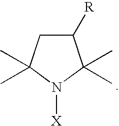

Wherein X is selected from O. and OH, and R is selected from COOH, CONH, CN, and CH₂NH₂.

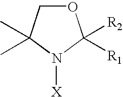

Wherein X is selected from O. and OH, and R₁ is selected from CH₃ and spirocyclohexyl, and R₂ is selected from C₂H₅ and spirocyclohexyl.

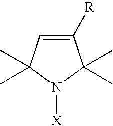

Wherein X is selected from O. and OH and R is selected from CONH.

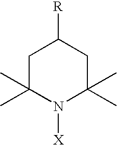

Wherein X is selected from O. and OH and R is selected from H, OH, and NH₂.

Suitable nitroxide compounds can also be found in Proctor, U.S. Pat. No. 5,352,442, and Mitchell et al., U.S. Pat. No. 5,462,946, both of which are hereby incorporated by reference in their entireties.

A non-limiting list of nitroxide compounds include: 2-ethyl-2,5,5-trimethyl-3-oxazolidine-1-oxyl (OXANO), 2,2,6,6-tetramethylpiperidine-1-oxyl (TEMPO), 4-hydroxy-2,2,6,6-tetramethylpiperidine-1-oxyl (TEMPOL), 4-amino-2,2,6,6-tetramethyl-1-piperidinyloxy (Tempamine), 3-Aminomethyl-PROXYL, 3-Cyano-PROXYL, 3-Carbamoyl-PROXYL, 3-Carboxy-PROXYL, and 4-Oxo-TEMPO. TEMPO can also be substituted, typically in the 4 position, for example, 4-amino, 4-(2-bromoacetamido), 4-(ethoxyfluorophosphonyloxy), 4-hydroxy, 4-(2-iodoacetamido), 4-isothiocyanato, 4-maleimido, 4-(4-nitrobenzoyloxyl), 4-phosphonooxy, and the like.

### Experimental Protocol

In a gene expression study, Tempol was administered to experimental mice at a dose of 5 g/kg of diet from 12 months through 15 months. Mice receiving the same diet without the addition of Tempol were used as a negative control. At the age of 15 months, the white adipose tissue of the experimental animals was obtained. The expression of a broad spectrum of genes in the adipose tissue was assessed using chip-based microarray technology. Specifically, in this case an Affymetrix MOE430A 2.0 array, containing 12,960 genes, was employed. Such chips are well known in the art and are widely used to assess gene expression. The experimental results showed that three genes encoding enzymes in the cholesterol synthesis pathway, lanosterol synthase (“LSS”), farnesyl diphosphate synthase (“FDPS”), and NAD(P) dependent steroid dehydrogenase-like (NSDHL), exhibited a decrease in expression. These genes are shown in Table 1.

TABLE 1
HYPERCHOLESTEROLEMIA-RELATED GENES
EXHIBITING DECREASED EXPRESSION IN ADIPOSE TISSUE
AFTER TEMPOL ADMINISTRATION

| | Mean | Mean | T-test | Fold |
|---|---|---|---|---|
| Description | (Control mice) | (Tempol-treated mice) | Value | change |
| LSS | 514 | 224 | 0 | −2.33 |
| FDPS | 2644 | 1274 | 0 | −2.08 |
| NSDHL | 2289 | 1398 | 0 | −1.64 |

A short summary of the genes described in Table 1 is provided below.

### Lanosterol Synthase (LSS)

Lanosterol synthase, also known as 2,3-epoxysqualene lanosterol-cyclase or 2,3-oxidosqualene cyclase, is involved in producing the characteristic ring structures of steroids such as cholesterol by converting (S)-2,3-epoxysqualene to lanosterol. In FIG. 1, the enzyme accordingly operates in the region marked (4), downstream of mevalonate synthesis.

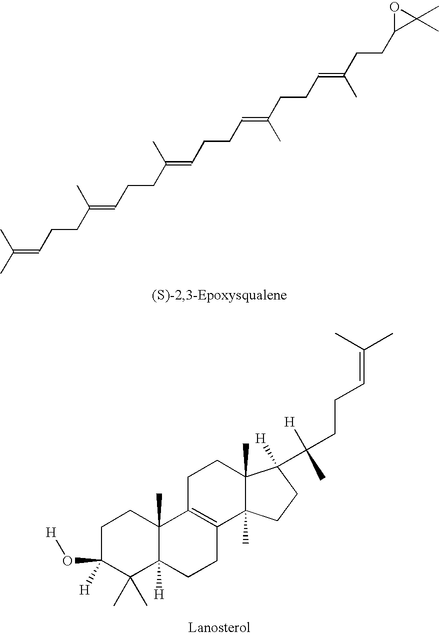

As shown in Table 1, the expression of LSS in the adipose tissue of the experimental mice was decreased 2.33-fold in the animals treated with Tempol.

### Farnesyl Diphosphate Synthase (FDPS)

Farnesyl diphosphate synthase, also known as dimethylallyltranstransferase, catalyzes two sequential 1′-4 condensation reactions of isopentenyl diphosphate (IPP) with the allelic diphosphates, dimethylallyl diphosphate, and geranyl diphosphate. The product of these enzymatic reactions, farnesyl diphosphate, is a precursor to squalene, an early step in the cholesterol synthesis pathway. Accordingly, this enzyme operates in the region marked (3) (and thus downstream of mevalonate synthesis) in FIG. 1.

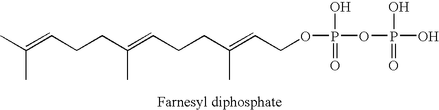

As shown in Table 1, the expression of FDPS in the adipose tissue of the experimental mice was decreased 2.08-fold in the animals treated with Tempol.

### NAD(P) Dependent Steroid Dehydrogenase-Like (NSDHL)

NAD(P) dependent steroid dehydrogenase-like, also known as NAD(P)-dependent steroid dehydrogenase or H105e3, encodes a sterol dehydrogenase or decarboxylase involved in the sequential removal of two C-4 methyl groups in post-squalene cholesterol biosynthesis. This is carried out in section (4) of FIG. 1, and further downstream in the synthesis pathway than lanosterol synthase described above. Specifically, NSDHL first converts 4α-carboxy-4α-methyl-5α-cholesta-8,24-dien-3β-ol to 4α-methyl-5α-cholesta-8,24-dien-3-one, and at a later point in the pathway converts 4α-carboxy-5α-cholesta-8,24-dien-3β-ol to 5α-cholesta-8,24-dien-3-one.

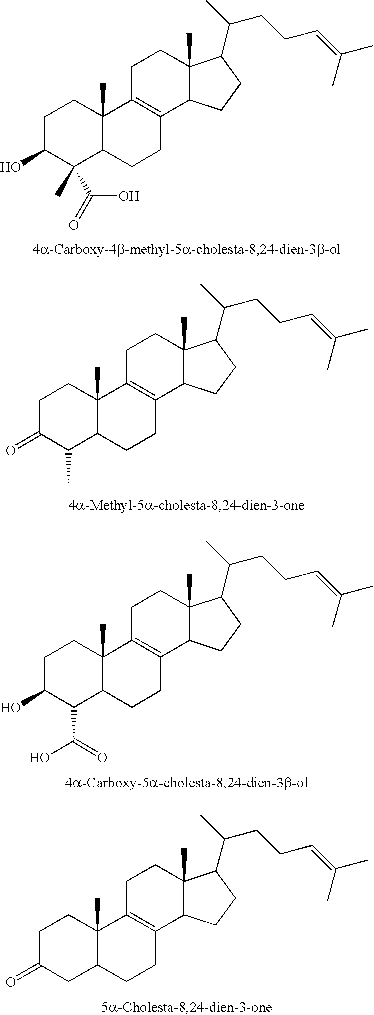

As shown in Table 1, the expression of NSHDL in the adipose tissue of the experimental mice was decreased 1.64-fold in the animals treated with Tempol.

### Preferred Embodiment: Cardiovascular Disease Prophylaxis and Treatment Protocol

As described above, Tempol has the effect of altering the expression of genes related to hypercholesterolemia. Since the expression of these genes is altered, administration of Tempol will have a beneficial effect by altering concentrations of gene products that are linked to the amelioration of hypercholesterolemia. Specifically, Tempol will have at least the beneficial effects of reducing the concentration of LSS, FDPS, and NSDHL, thereby reducing the rate at which cholesterol is synthesized and ultimately the amount of cholesterol present in the blood. Furthermore, because Tempol is not known to affect the levels of enzymes in the mevalonate pathway, it will be possible to reduce the amount of de novo synthesis of cholesterol without affecting levels of mevalonate, thus avoiding the undesirable side effects associated with statin-induced reductions in mevalonate synthesis.

In a preferred embodiment of the present invention, therefore, Tempol is administered to a mammalian host, such as a human, exhibiting no symptoms of hypercholesterolemia in order to prevent the development of hypercholesterolemia. Particularly preferred patients are those who are predisposed or otherwise at risk for hypercholesterolemia, such as those with a family history of cardiovascular disease or those with genetic or serum markers associated with hypercholesterolemia. Alternatively, Tempol may be administered to a human exhibiting hypercholesterolemia, in order to reduce the severity of hypercholesterolemia or return HDL and LDL levels to within normal ranges. For this purpose, Tempol, non-toxic salts thereof, acid addition salts thereof or hydrates thereof may be administered systemically or locally, usually by oral or parenteral administration.

The doses to be administered are determined depending upon, for example, age, body weight, symptom, the desired therapeutic effect, the route of administration, and the duration of the treatment. In the human adult, the dose per person at a time is generally from about 0.01 to about 1000 mg, by oral administration, up to several times per day. Specific examples of particular amounts contemplated via oral administration include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000 or more mg. The dose per person at a time is generally from about 0.01 to about 300 mg/kg via parenteral administration (preferably intravenous administration), from one to four or more times per day. Specific examples of particular amounts contemplated include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 or more mg/kg. Continuous intravenous administration is also contemplated for from 1 to 24 hours per day to achieve a target concentration from about 0.01 mg/L to about 100 mg/L. Specific examples of particular amounts contemplated via this route include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more mg/L. The dose to be used does, however, depend upon various conditions, and there may be cases wherein doses lower than or greater than the ranges specified above are used.

Tempol may be administered in the form of, for example, solid compositions, liquid compositions or other compositions for oral administration, injections, liniments or suppositories for parenteral administration.

Solid compositions for oral administration include compressed tablets, pills, capsules, dispersible powders and granules. Capsules include hard capsules and soft capsules. In such solid compositions, Tempol may be admixed with an excipient (e.g. lactose, mannitol, glucose, microcrystalline cellulose, starch), combining agents (hydroxypropyl cellulose, polyvinyl pyrrolidone or magnesium metasilicate aluminate), disintegrating agents (e.g. cellulose calcium glycolate), lubricating agents (e.g. magnesium stearate), stabilizing agents, agents to assist dissolution (e.g. glutamic acid or aspartic acid), or the like. The agents may, if desired, be coated with coating agents (e.g. sugar, gelatin, hydroxypropyl cellulose or hydroxypropylmethyl cellulose phthalate), or be coated with two or more films. Further, coating may include containment within capsules of absorbable materials such as gelatin.

Liquid compositions for oral administration include pharmaceutically acceptable solutions, suspensions, emulsions, syrups and elixirs. In such compositions, Tempol is dissolved, suspended or emulsified in a commonly used diluent (e.g. purified water, ethanol or mixture thereof). Furthermore, such liquid compositions may also comprise wetting agents or suspending agents, emulsifying agents, sweetening agents, flavoring agents, perfuming agents, preserving agents, buffer agents, or the like.

Injections for parenteral administration include solutions, suspensions, emulsions and solids which are dissolved or suspended. In injections, Tempol may be dissolved, suspended and emulsified in a solvent. The solvents are, for example, distilled water for injection, physiological salt solution, vegetable oil, propylene glycol, polyethylene glycol, alcohol such as ethanol, or a mixture thereof. Moreover the injections may also include stabilizing agents, agents to assist dissolution (e.g. glutamic acid, aspartic acid or POLYSORBATE80 (registered trade mark)), suspending agents, emulsifying agents, soothing agents, buffer agents, preserving agents, etc. They are sterilized in the final process or manufactured and prepared by sterile procedure. They may also be manufactured in the form of sterile solid compositions, such as a freeze-dried composition, and they may be sterilized or dissolved immediately before use in sterile distilled water for injection or some other solvent.

Other compositions for parenteral administration include liquids for external use, and ointment, endermic liniments, inhale, spray, suppositories for rectal administration and pessaries for vaginal administration which comprise Tempol and are administered by methods known in the art.

Spray compositions may comprise additional substances other than diluents: e.g. stabilizing agents (e.g. sodium sulfite hydride), isotonic buffers (e.g. sodium chloride, sodium citrate or citric acid). For preparation of such spray compositions, for example, the method described in U.S. Pat. No. 2,868,691 or U.S. Pat. No. 3,095,355 may be used. Briefly, a small aerosol particle size useful for effective distribution of the medicament may be obtained by employing self-propelling compositions containing the drugs in micronized form dispersed in a propellant composition. Effective dispersion of the finely divided drug particles may be accomplished with the use of very small quantities of a suspending agent, present as a coating on the micronized drug particles. Evaporation of the propellant from the aerosol particles after spraying from the aerosol container leaves finely divided drug particles coated with a fine film of the suspending agent. In the micronized form, the average particle size is less than about 5 microns. The propellant composition may employ, as the suspending agent, a fatty alcohol such as oleyl alcohol. The minimum quantity of suspending agent is approximately 0.1 to 0.2 percent by weight of the total composition. The amount of suspending agent is preferably less than about 4 percent by weight of the total composition to maintain an upper particle size limit of less than 10 microns, and preferably 5 microns. Propellants that may be employed include hydrofluoroalkane propellants and chlorofluorocarbon propellants. Dry powder inhalation may also be employed.

Furthermore, the use of a nitroxide antioxidant in the preparation of a medicament for altering intracellular levels of one or more proteins associated with hypercholesterolemia, the use of a nitroxide antioxidant in the preparation of a medicament for inhibiting the progression of hypercholesterolemia associated with a protein, the use of a nitroxide antioxidant in the preparation of a medicament for treating hypercholesterolemia, and the use of a nitroxide antioxidant in the preparation of a medicament for reducing fasting plasma total cholesterol, are all specifically contemplated. In preferred embodiments of these uses, the nitroxide antioxidant is Tempol.

### Example 1

A 70-kilogram patient having levels of fasting plasma total cholesterol indicative of hypercholesterolemia is administered a dose of 1500 mg of Tempol per day for 180 days. This may be administered in a single dose, or may be administered as a number of smaller doses over a 24-hour period: for example, three 500-mg doses at eight-hour intervals. Following treatment, the protein level of each of LSS, FDPS, and NSDHL is reduced.

### Example 2

A 70-kilogram patient at risk for developing hypercholesterolemia is administered a dose of 1500 mg of Tempol per day for 180 days. This may be administered in a single dose, or may be administered as a number of smaller doses over a 24-hour period: for example, three 500-mg doses at eight-hour intervals. Following treatment, the protein level of each of LSS, FDPS, and NSDHL is reduced.

1. A method for altering intracellular levels of one or more proteins associated with hypercholesterolemia, comprising:
identifying an individual in need of altering levels of hypercholesterolemia-associated proteins; and
administering to that individual an effective amount of a nitroxide antioxidant.

2. The method of claim 1 wherein the nitroxide antioxidant is selected from the group consisting of 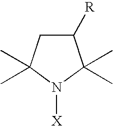
or a pharmaceutically acceptable salt thereof
wherein X is selected from O. and OH, and R is selected from COOH, CONH, CN, and CH₂ NH₂; 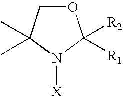
or a pharmaceutically acceptable salt thereof
wherein X is selected from O. and OH, and R₁ is selected from CH₃ and spirocylohexyl, and R₂ is selected from C₂H₅ and spirocyclohexyl; 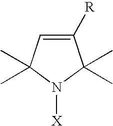
or a pharmaceutically acceptable salt thereof
wherein X is selected from O. and OH and R is CONH; 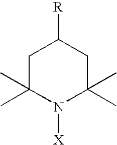
or a pharmaceutically acceptable salt thereof
wherein X is selected from O. and OH and R is H, OH, and NH₂; 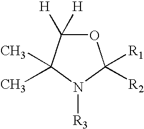
wherein R₁ is —CH₃; R₂ is —C₂H₅, —C₃H₇, —C₄H₉, —C₅H₁₁, —C₆H₁₃, —CH₂—CH(CH₃)₂, —CHCH₃C₂H₅, or —(CH₂)₇—CH₃, or wherein R₁ and R₂ together form spirocyclopentane, spirocyclohexane, spirocycloheptane, spirocyclooctane, 5-cholestane, or norbornane; R₃ is —O. or —OH, or a physiologically acceptable salt thereof which has antioxidant activity; 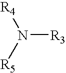
wherein R₃ is —O. or —OH; and
wherein R₄ and R₅ combine together with the nitrogen to form a heterocyclic group; wherein the atoms in the heterocyclic group (other than the N atom shown in the formula) may be all C atoms or may be C atoms and one or more N, O and/or S atoms; or
wherein R₄ and R₅ combine together to form substituted or unsubstituted pyrrole, imidazole, oxazole, thiazole, pyrazole, 3-pyrroline, pyrrolidine, pyridine, pyrimidine, or purine; or
wherein R₄ and R₅ themselves comprise a substituted or unsubstituted cyclic or heterocyclic group;
2-ethyl-2,5,5-trimethyl-3-oxazolidine-1-oxyl, 2,2,6,6-tetramethylpiperidine-1-oxyl (TEMPO), 4-hydroxy-2,2,6,6-tetramethylpiperidine-1-oxyl (TEMPOL), 4-amino-2,2,6,6-tetramethyl-1-piperidinyloxy (Tempamine), 3-Aminomethyl-PROXYL, 3-Cyano-PROXYL, 3-Carbamoyl-PROXYL, 3-Carboxy-PROXYL, 4-oxo-TEMPO, 4-amino-TEMPO, 4-(2-bromoacetamido)-TEMPO, 4-(ethoxyfluorophosphonyloxy-TEMPO, 4-hydroxy-TEMPO, 4-(2-iodoacetamido)-TEMPO, 4-isothiocyanato-TEMPO, 4-maleimido-TEMPO, 4-(4-nitrobenzoyloxyl)-TEMPO, and 4-phosphonooxy-TEMPO.

3. The method of claim 1, wherein the nitroxide antioxidant is 4-hydroxy-2,2,6,6-tetramethylpiperidine-1-oxyl.

4. The method of claim 1, wherein the level of the hypercholesterolemia associated protein is decreased.

5. The method of claim 4, wherein the hypercholesterolemia associated protein is LSS, FDPS, or NSDHL.

6. The method of claim 1, wherein the effective amount of a nitroxide antioxidant is within a range of 0.01-300 mg/kg.

7. The method of claim 1, wherein the effective amount of a nitroxide antioxidant is within a range of 0.1-250 mg/kg.

8. The method of claim 1, wherein the effective amount of a nitroxide antioxidant is within a range of 1-200 mg/kg.

9. The method of claim 1, wherein the effective amount of a nitroxide antioxidant is within a range of 2-150 mg/kg.

10. The method of claim 1, wherein the effective amount of a nitroxide antioxidant is within a range of 5-125 mg/kg.

11. The method of claim 1, wherein the effective amount of a nitroxide antioxidant is within a range of 7-100 mg/kg.

12. The method of claim 1, wherein the effective amount of a nitroxide antioxidant is within a range of 10-75 mg/kg.

13. The method of claim 1, wherein the effective amount of a nitroxide antioxidant is within a range of 15-30 mg/kg.

14. A method for inhibiting the progression of hypercholesterolemia associated with a protein, comprising:
identifying an individual affected by or at risk for the protein-associated hypercholesterolemia; and
administering to that individual an amount of a nitroxide antioxidant effective to alter expression of a gene associated with the protein-associated hypercholesterolemia.

15. The method of claim 14 wherein the nitroxide is selected from the group consisting of 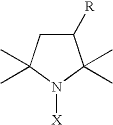
or a pharmaceutically acceptable salt thereof
wherein X is selected from O. and OH, and R is selected from COOH, CONH, CN, and CH₂NH₂; 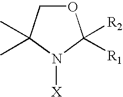
or a pharmaceutically acceptable salt thereof
wherein X is selected from O. and OH, and R₁ is selected from CH₃ and spirocylohexyl, and R₂ is selected from C₂H₅ and spirocyclohexyl; 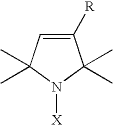
or a pharmaceutically acceptable salt thereof
wherein X is selected from O. and OH and R is CONH; 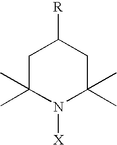
or a pharmaceutically acceptable salt thereof
wherein X is selected from O. and OH and R is H, OH, and NH₂; 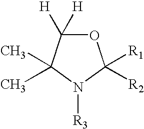
wherein R₁ is —CH₃; R₂ is —C₂H₅, —C₃H₇, —C₄H₉, —C₅H₁₁, —C₆H₁₃, —CH₂CH(CH₃)₂, —CHCH₃C₂H₅, or —(CH₂)₇—CH₃, or wherein R₁ and R₂ together form spirocyclopentane, spirocyclohexane, spirocycloheptane, spirocyclooctane, 5-cholestane, or norbornane; R₃ is —O. or —OH, or a physiologically acceptable salt thereof which has antioxidant activity; 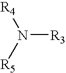
wherein R₃ is —O. or —OH; and
wherein R₄ and R₅ combine together with the nitrogen to form a heterocyclic group; wherein the atoms in the heterocyclic group (other than the N atom shown in the formula) may be all C atoms or may be C atoms and one or more N, O and/or S atoms; or
wherein R₄ and R₅ combine together to form substituted or unsubstituted pyrrole, imidazole, oxazole, thiazole, pyrazole, 3-pyrroline, pyrrolidine, pyridine, pyrimidine, or purine; or
wherein R₄ and R₅ themselves comprise a substituted or unsubstituted cyclic or heterocyclic group;
2-ethyl-2,5,5-trimethyl-3-oxazolidine-1-oxyl, 2,2,6,6-tetramethylpiperidine-1-oxyl (TEMPO), 4-hydroxy-2,2,6,6-tetramethylpiperidine-1-oxyl (TEMPOL), 4-amino-2,2,6,6-tetramethyl-1-piperidinyloxy (Tempamine), 3-Aminomethyl-PROXYL, 3-Cyano-PROXYL, 3-Carbamoyl-PROXYL, 3-Carboxy-PROXYL, 4-oxo-TEMPO, 4-amino-TEMPO, 4-(2-bromoacetamido) -TEMPO, 4-(ethoxyfluorophosphonyloxy)-TEMPO, 4-hydroxy-TEMPO, 4-(2-iodoacetamido)-TEMPO, 4-isothiocyanato-TEMPO, 4-maleimido-TEMPO, 4-(4-nitrobenzoyloxyl) -TEMPO, and 4-phosphonooxy-TEMPO.

16. The method of claim 14, where the expression of the gene is decreased.

17. The method of claim 15, wherein the gene is LSS, FDPS, or NSDHL.

18. The method of claim 16 wherein the nitroxide antioxidant is 4-hydroxy-2,2,6,6-tetramethylpiperidine-1-oxyl.

19. The method of claim 14, wherein the effective amount of a nitroxide antioxidant is within a range of 0.01-300 mg/kg.

20. The method of claim 15, wherein the effective amount of a nitroxide antioxidant is within a range of 0.1-250 mg/kg.

21. The method of claim 14, wherein the effective amount of a nitroxide antioxidant is within a range of 1-200 mg/kg.

22. The method of claim 14, wherein the effective amount of a nitroxide antioxidant is within a range of 2-150 mg/kg.

23. The method of claim 14, wherein the effective amount of a nitroxide antioxidant is within a range of 5-125 mg/kg.

24. The method of claim 14, wherein the effective amount of a nitroxide antioxidant is within a range of 7-100 mg/kg.

25. The method of claim 14, wherein the effective amount of a nitroxide antioxidant is within a range of 10-75 mg/kg.

26. The method of claim 14, wherein the effective amount of a nitroxide antioxidant is within a range of 15-30 mg/kg.

27. A method for treating hypercholesterolemia, comprising:
administering to a patient with hypercholesterolemia an amount of a nitroxide antioxidant effective to reduce fasting plasma total cholesterol.

28. The method of claim 27 wherein the nitroxide is selected from the group consisting of 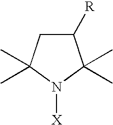
or a pharmaceutically acceptable salt thereof
wherein X is selected from O. and OH, and R is selected from COOH, CONH, CN, and CH₂NH₂; 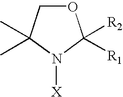
or a pharmaceutically acceptable salt thereof
wherein X is selected from O. and OH, and R₁ is selected from CH₃ and spirocylohexyl, and R₂ is selected from C₂H₅ and spirocyclohexyl; 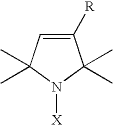
or a pharmaceutically acceptable salt thereof
wherein X is selected from O. and OH and R is CONH; 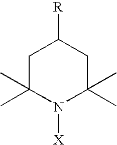
or a pharmaceutically acceptable salt thereof
wherein X is selected from O. and OH and R is H, OH, and NH₂; 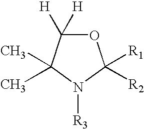
wherein R₁ is —CH₃; R₂ is —C₂H₅, —C₃H₇, —C₄H₉, —C₅H₁₁, —C₆H₁₃, —CH₂—CH(CH₃)₂, —CHCH₃C₂H₅, or —(CH₂)₇—CH₃, or wherein R₁ and R₂ together form spirocyclopentane, spirocyclohexane, spirocycloheptane, spirocyclooctane, 5-cholestane, or norbornane; R₃ is —O. or —OH, or a physiologically acceptable salt thereof which has antioxidant activity; 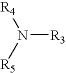
wherein R₃ is —O. or —OH; and
wherein R₄ and R₅ combine together with the nitrogen to form a heterocyclic group; wherein the atoms in the heterocyclic group (other than the N atom shown in the formula) may be all C atoms or may be C atoms and one or more N, O and/or S atoms; or
wherein R₄ and R₅ combine together to form substituted or unsubstituted pyrrole, imidazole, oxazole, thiazole, pyrazole, 3-pyrroline, pyrrolidine, pyridine, pyrimidine, or purine; or
wherein R₄ and R₅ themselves comprise a substituted or unsubstituted cyclic or heterocyclic group;
2-ethyl-2,5,5-trimethyl-3-oxazolidine-1-oxyl, 2,2,6,6-tetramethylpiperidine-1-oxyl (TEMPO), 4-hydroxy-2,2,6,6-tetramethylpiperidine-1-oxyl (TEMPOL), 4-amino-2,2,6,6-tetramethyl-1-piperidinyloxy (Tempamine), 3-Aminomethyl-PROXYL, 3-Cyano-PROXYL, 3-Carbamoyl-PROXYL, 3-Carboxy-PROXYL, 4-oxo-TEMPO, 4-amino-TEMPO, 4-(2-bromoacetamido)-TEMPO, 4-(ethoxyfluorophosphonyloxy)-TEMPO, 4-hydroxy-TEMPO, 4-(2-iodoacetamido)-TEMPO, 4-isothiocyanato-TEMPO, 4-maleimido-TEMPO, 4-(4-nitrobenzoyloxyl)-TEMPO, and 4-phosphonooxy-TEMPO.

29. The method of claim 27, wherein the effective amount of a nitroxide antioxidant is within a range of 0.01-300 mg/kg.

30. The method of claim 27, wherein the effective amount of a nitroxide antioxidant is within a range of 0.1-250 mg/kg.

31. The method of claim 27, wherein the effective amount of a nitroxide antioxidant is within a range of 1-200 mg/kg.

32. The method of claim 27, wherein the effective amount of a nitroxide antioxidant is within a range of 10-75 mg/kg.

33. The method of claim 27, wherein the effective amount of a nitroxide antioxidant is within a range of 15-30 mg/kg.

34-37. (canceled)

38. A method to return HDL levels to normal ranges comprising
identifying an individual with abnormal HDL levels; and
administering to that individual and effective amount of a nitroxide antioxidant.

39. The method of claim 38 wherein the nitroxide antioxidant is 4-hydroxy-2,2,6,6-tetramethyl-1-piperidinyloxyl.
